(19) United States
(12) Patent Application Publication
(10) Pub. No.: US 2015/0374527 A1
(43) Pub. Date: Dec. 31, 2015

(54) ANKLE-FOOT ORTHOTIC FOR TREATMENT OF FOOT DROP

(71) Applicant: JE3.LLC, St. Paul, MN (US)

(72) Inventor: Jerry A. Wenger, Powell, WY (US)

(21) Appl. No.: 14/754,830

(22) Filed: Jun. 30, 2015

Related U.S. Application Data
(60) Provisional application No. 62/019,060, filed on Jun. 30, 2014.

(51) Int. Cl. A61F 5/01

(52) U.S. Cl. CPC A61F 5/0113

(57) ABSTRACT

An ankle-foot orthotic is provided that treats steppage gate by preventing foot drop during movement. The orthotic having an ankle brace linked to a user's shoe by an elastic strap. The elastic strap having sufficient elasticity to provide the appropriate amount of tensile force to the shoe to prevent foot drop, while having sufficient flexibility to allow the user to naturally flex their foot and ankle during movement. The elastic strap comprises hook fasteners attachable to and removable from the shoe, such that the orthotic can be easily put on or taken off by the user.

### RELATED APPLICATION

The present application claims the benefit of U.S. Provisional Application No. 62/019,060 filed Jun. 30, 2014, which is hereby incorporated herein in its entirety by reference.

### FIELD OF THE INVENTION

The invention relates generally to treating steppage gait in persons suffering from a foot drop condition, and more specifically, to an apparatus and related methods for supporting the front portion of the foot to prevent foot drop and alleviate the steppage gait caused by the foot drop.

### BACKGROUND OF THE INVENTION

Foot drop, also commonly known as drop foot, is a common condition in which a person is unable to lift the front portion of one or both feet during walking or running due to a neurological or muscular condition which weakens the muscles of the foot. The condition causes the front portion of the person's foot to slap down on the floor or drag across the floor during walking or running. Persons suffering from foot drop often compensate by lifting the afflicted foot higher than normally necessary when moving to prevent the dropping foot from dragging or slapping on the floor. The irregular gait caused by the foot drop and the compensating movement by the person is known as a steppage gait.

Foot drop is typically treated by ankle-foot orthotics that either lock the person's ankle in place or support the front portion of the person's foot. However, the currently available ankle-foot orthotics often restrict the natural flexing motion of the ankle and foot during normal movement to prevent the foot drop motion, replacing the steppage gait with a different irregular gait. Ankle locking orthotics, such as the one described in U.S. Pat. No. 3,916,886, typically comprise rigid L-shaped members fitted against the back of the ankle and the underside of the foot to prevent the entire foot from flexing downwardly at the ankle U.S. Pat. No. 3,916,886 is hereby incorporated by reference in its entirety. However, because the L-shape member completely prevents downward flexing of the ankle at the foot, the foot cannot make the slight downward flexing movement that naturally occurs during walking or running. Ankle-foot orthotics that provide support to the front portion of the foot also impede the natural flexing of the foot and ankle during a natural gait. These orthotics typically comprise straps anchored to the person's calf or ankle at one end and anchored to the person's foot or shoe at the other end as disclosed in U.S. Pat. Nos. 4,817,589 and 7,458,950, to provide a tensile force preventing downward motion of the foot. U.S. Pat. Nos. 4,817,589 and 7,458,950 are also hereby incorporated by reference in their entirety. However, the straps used are often static or only have a limited elasticity to ensure sufficient tensile force is applied to support the front portion of the foot. The inelasticity of the straps also prevents the natural flexing of the foot and ankle during normal movement. The limited flexibility of presently available ankle foot orthotics effectively replace the irregular steppage gait with an irregular gait

In addition to being overly restrictive, currently available ankle-foot orthotics are often too bulky to fit easily into shoes. Similarly, currently available ankle-foot orthotics often require numerous straps or anchors to properly secure the orthotic to the person's ankle or foot increasing the difficulty of putting the orthotic on or taking it off. For example, the L-shaped members of ankle locking orthotics are often large and do not easily fit into shoes without extensive modification of the shoe. In addition, the rigidness of the L-shape member prevents wearers from pointing their toes to ease put on or takeoff the shoe. Similarly, strap orthotics often employ a plurality of straps and anchors that must be attached to the shoe or foot to properly attach the orthotic to the person's ankle and foot. The straps and the anchors may be difficult to remove from the shoe if the person wants to remove or change shoes. The complexity and the difficulty of putting on and taking off the currently available orthotics often cause wearers to spend considerable time putting on or taking off orthotic or forgoing the orthotic altogether.

An additional problem for strap orthotics is the positioning of the straps and locating where the straps are anchored on the wearer. Typically, the anchor locations for the straps are placed as high up on the calf as possible and as far down the foot or shoe as possible to achieve the maximum tensile force. However, the higher up the calf or further down the foot the anchor points are placed, the more visible and cumbersome the orthotic becomes for the user. Similarly, the greater the number of straps, the more support is provided for the foot. As with the positioning of the anchor locations, the greater the number of straps, the more visible and cumbersome the orthotic becomes for the wearer. Similarly, the positioning of the straps may be uncomfortable for users if the straps are pressed against the person's foot or ankle. The straps may rub against the person's legs or feet during movement causing chaffing or other discomfort.

### SUMMARY OF THE INVENTION

An embodiment of the present invention is directed to an ankle-foot orthotic for applying a pulling force to a wearer's shoe to counteract the effects of drop foot and prevent steppage gait. More specifically, the ankle-foot orthotic anchors the top of the wearer's shoe to an ankle brace around the wearer's ankle with an elastic strap. The elastic strap applies a tensile force to the top of the wearer's shoe to prevent steppage gait while still providing the necessary flexibility to allow the wearer to flex their foot and ankle naturally during walking or running. The ankle-foot orthotic also comprises a highly customizable engagement system for linking the orthotic to the wearer's shoe allowing the wearer to customize where the tensile force is directed and the amount of tensile force applied.

An ankle-foot orthotic for treating steppage gait according to an embodiment of the present invention generally comprises an ankle brace and an elastic strap. The ankle brace further comprises wings for wrapping around a wearer's ankle and also defines an exterior face and an interior face contacting the wearer's ankle. In an embodiment of the present invention, an ankle pad can be positioned between the wearer's ankle and interior face of the ankle brace to prevent discomfort to the wearer while wearing the ankle-foot orthotic. A primary strap loop for receiving the elastic strap is positioned on the exterior face of the ankle brace between the first and second wings. In operation, wrapping the ankle brace around the wearer's ankle positions the primary strap loop at the rear of the wearer's ankle.

The elastic strap defines a first end and a second end and is adapted to engage the wearer's shoe. The elastic strap can further comprise a hook fastener disposed at each end for engaging any looped structures on the wearer's shoes such as shoe laces or eyelets for receiving shoe laces. The hook fasteners allow for easy engagement of the shoe and customization as to the exact location that the tensile force provided by the elastic strap will be applied. Alternatively, the elastic strap can further comprise a strap adjustment mechanism having a first buckle and a second buckle positioned on the elastic strap. In this configuration, the first and second ends of the elastic strap are threaded through the looped structure on the shoe and looped back to the first and second buckle respectively. The strap adjustment mechanism allows the wearer to change the effective length of the elastic strap and correspondingly the tension applied by the elastic strap to the wearer's shoe.

According to an embodiment of the present invention, in operation, the elastic strap is threaded through the primary strap loop and wrapped around the wearer's ankle to engage the wearer's shoe. In this arrangement, the elastic strap is angled at an ideal angle and providing the correct tension force for maintaining the wearer's shoe in the correct position. Alternatively, the ankle brace can be wrapped around the wearer's ankle such that the primary strap loop is disposed at the front of the wearer's angle. In this configuration, the elastic strap is not wrapped around the wearer's ankle and instead directly engages the wearer's shoe.

According to an embodiment of the present invention, the ankle-foot orthotic can further comprise ring anchors engageable to any looped structure on the wearer's shoe, including the shoe lace eyelets. The ring anchors can be easily engaged by or disengaged from the hook fasteners of the elastic strap. Consequently, the ring anchors can significantly reduce the time necessary to put on and take off the ankle-foot orthotic as well as simply the customization of the ankle foot orthotic allowing wearers to quickly try out a plurality of positions and arrangements for engaging the wearer's shoe with the elastic strap.

According to an embodiment of the invention, the orthotic can further comprise a support fitted to the exterior of the ankle brace. The support is adapted to protect the rear of the user's ankle from chaffing or other discomfort from the elastic strap rubbing against the user's ankle during movement. The ankle support can comprise a rigid polymer to provide support for the user's ankle and preventing the user from feeling any discomfort from the shifting or stretching of the elastic strap while walking. The ankle support is countered to protect the user's ankle without inhibiting the user's flexibility so as to allow the user to walk without hindrance.

According to an embodiment of the present invention, the support can further comprise a strap adjustment assembly having an adjustable primary strap loop and two secondary strap loops positioned within cutouts in the support. The elastic strap can be threaded through the primary and secondary strap loops to form a u-shaped portion in the elastic strap. The length of the adjustable primary strap loop can be adjusted to increase or decrease the size of the u-shaped portion, which correspondingly changes the effective length of the elastic strap and the tension applied to the wearer's shoe. Alternatively, the strap adjustment assembly can comprise a fixed length primary strap loop and two secondary strap loops. In this configuration, the support comprises a plurality of cutouts for receiving the primary strap loop. Positioning the primary strap loop in the various cutouts changes the size of the u-shaped portion and correspondingly the tension applied to the wearer's shoe.

According to an embodiment of the present invention, a fastener clip can comprise an anchor portion, a first longitudinal side portion, a curved end portion, a second longitudinal side portion, and an end portion. The fastener clip can be inserted and removed from an eyelet and can have a structure capable of anchoring the fastener clip in a proper position while allowing rotation and movement of fastener clip. A hook fastener can engage with the curved end portion to provide tension force.

According to an embodiment, an ankle brace can be shaped to conform to the ankle area and have a built-in conformable support fixedly sandwiched between the interior face and the exterior face of the brace. The built-in conformable support can be comprised of a plurality of longitudinal support members of varying lengths and configurations and can be comprised of a stiff yet flexible material. The length of each individual longitudinal support member is chosen so that the ankle-foot orthotic can be form fitting and provide sufficient structure and support while allowing mobility of the ankle and reducing pressure points. A strap loop is fixedly attached to the built-in conformable support.

The above summary of the invention is not intended to describe each illustrated embodiment or every implementation of the present invention. The Figures and the Detailed Description that follow more particularly exemplify these embodiments.

### BRIEF DESCRIPTION THE DRAWINGS

The invention may be more completely understood in consideration of the following detailed description of various embodiments of the invention in connection with the accompanying drawings, in which:

FIG. 1 is a perspective view of an ankle-foot orthotic in an unrolled configuration presenting the exterior face of the orthotic according to an embodiment.

FIG. 2 is a perspective view of an ankle-foot orthotic in an unrolled configuration presenting the interior face of the orthotic according to an embodiment.

FIG. 3 is an exploded view of an ankle-foot orthotic according to an embodiment.

FIG. 4 is cross sectional view of an ankle-foot orthotic in an unrolled configuration according to an embodiment.

FIG. 5 is a front perspective view of an ankle-foot orthotic according to an embodiment wherein the ankle brace is being wrapped around a user's ankle.

FIG. 6 is a rear perspective view of an ankle-foot orthotic according to an embodiment wherein the ankle brace is being wrapped around a user's ankle.

FIG. 7 is a perspective view of an ankle-foot orthotic according to an embodiment wherein the hook fasteners are being attached to the shoe.

FIG. 8 is a front perspective view of a foot having an ankle-foot orthotic fully attached to the user's ankle and foot.

FIG. 9 is a rear perspective view of a foot having an ankle-foot orthotic fully attached to the user's ankle and foot.

FIG. 10 is rear perspective view of an ankle-foot orthotic according to an embodiment of the invention.

FIG. 11 is a rear perspective view of the ankle foot orthotic depicted in FIG. 10 with the elastic strap removed.

FIG. 12 is a side perspective view of the ankle foot orthotic depicted in FIG. 11.

FIG. 13 is a perspective view of the interior side of a support for the ankle foot orthotic.

FIG. 14 is a perspective view of the exterior side of the support depicted in FIG. 13.

FIG. 15 is a side view of an ankle foot orthotic for a slipper according to an embodiment of the present invention.

FIG. 16 is a top view of the slipper for the ankle foot orthotic depicted in FIG. 15.

FIG. 17 is a side view of the ankle brace for the ankle foot orthotic depicted in FIG. 17 in the open configuration.

FIG. 18 is a perspective view of the ankle foot depicted in FIG. 15 fully attached to the user's ankle and foot.

FIG. 19 is a perspective view of the ankle foot depicted in FIG. 15 fully attached to the user's ankle and foot.

FIG. 20 is a side view of the grommet assembly according to an embodiment of the present invention.

FIG. 21 is a rear perspective view of an elastic strap adjustment assembly according an embodiment of the present assembly.

FIG. 22 is a rear perspective view of the elastic strap adjustment assembly depicted in FIG. 21.

FIG. 23 is a rear perspective view of the elastic strap adjustment assembly depicted in FIG. 21.

FIG. 24 is a rear perspective view of the elastic strap adjustment assembly depicted in FIG. 21.

FIG. 25 is an exploded view of an ankle-foot orthotic having a tension adjustment assembly according to an embodiment.

FIG. 26 is a perspective view of the ankle brace depicted in FIG. 25.

FIG. 27 is a rear perspective view of the ankle-foot orthotic depicted in FIG. 25.

FIG. 28 illustrates a fastener clip according to an embodiment.

FIG. 29 illustrates a fastener clip in an operational position according to an embodiment.

FIG. 30 illustrates a fastener clip in an operational position according to an embodiment.

FIG. 31 illustrates an ankle-foot orthotic having a built-in conformable support fixedly sandwiched between the interior face and the exterior face of the brace according to an embodiment.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

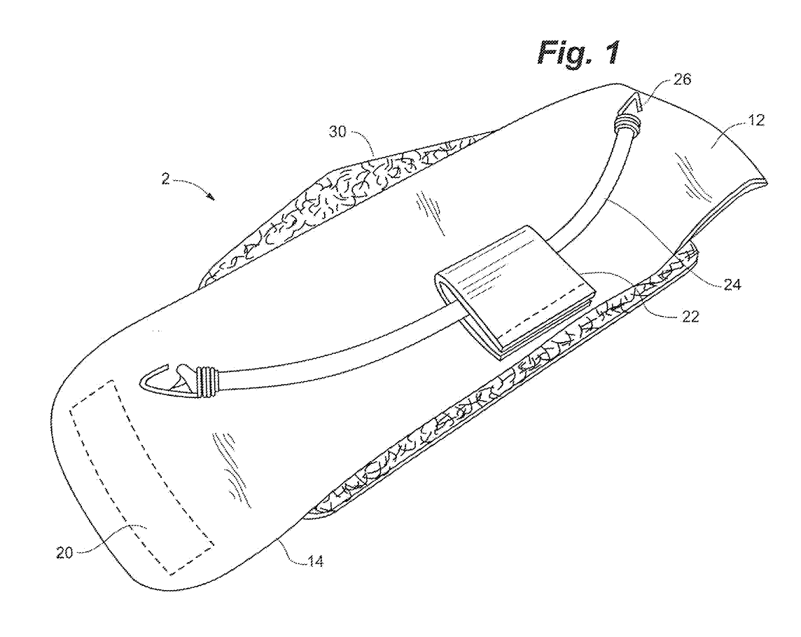

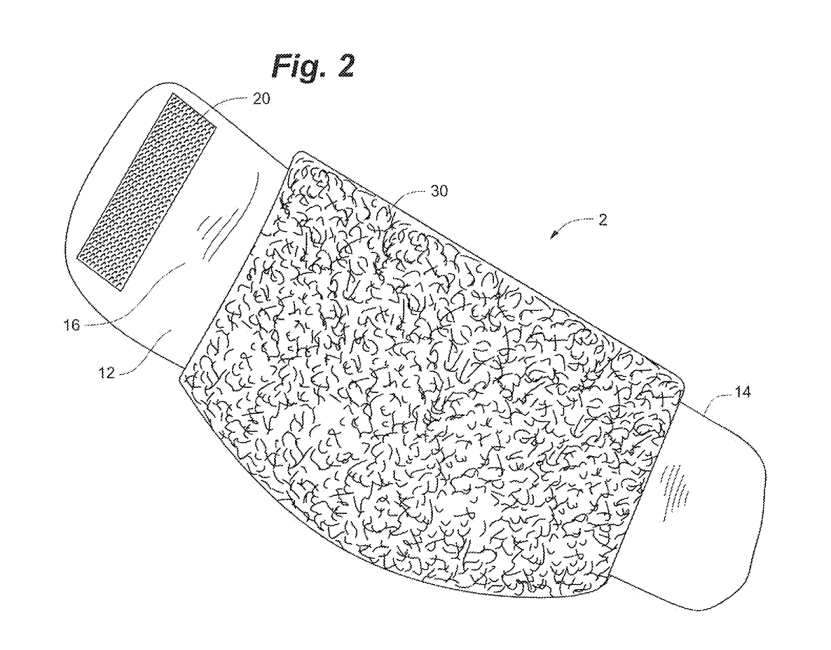

As illustrated in FIGS. 1-5, an ankle-foot orthotic 2 for treating steppage gait according to an embodiment of the present invention supports the weakened muscles by providing support beneath the wearer's foot or shoe 4 or by applying a pulling force to top of the wearer's shoe 4. The pulling force is typically provided by anchoring the front or top of wearer's foot or shoe 4 to the wearer's ankle 6.

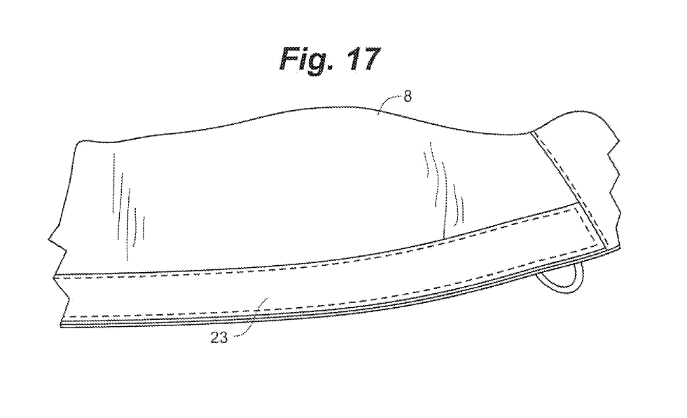

The ankle-foot orthotic 2, according to an embodiment of the present invention, generally comprises an ankle brace 8 and an elastic strap 24. The ankle brace 8 further comprises a first wing 12 and a second wing 14 and defines an interior face 16 and an exterior face 18. The ankle brace 8 further comprises an ankle brace fastener 20 for releasably engaging the first wing 12 to the second wing 14 such that the ankle brace 8 forms a generally cylindrical shape around a wearer's ankle 6. The ankle brace fastener 20 can comprise a VELCRO fastener, an elastic fastener, button fastener or any other temporary fastening means. Similarly, the ankle brace 8 can be elastic material adapted to pull against the wearer's ankle 6 to maintain the position of the ankle brace 8 on the wearer's ankle 6. A primary strap loop 22 is disposed on the exterior face 18 of the ankle brace 8 between the first and second wing 12, 14. According to an embodiment of the present invention, the ankle brace 8 can further comprise a structural strap 23 for maintaining the shape of the ankle brace 8 during movement and when tension force is applied to the ankle brace 8. The structural strap 23, as shown in FIG. 17, comprises a flexible plastic material sufficiently rigid to withstand the tension forces applied to the ankle brace 8, but also sufficiently flexible to wrap around the user's ankle 6.

The elastic strap 24 defines a first end 27 and second end 28. The elastic strap 24 is threadable through the primary strap loop 22 and comprises an elastic material for applying a pulling force to the wearer's shoe 4. According to an embodiment of the present invention, different elastic straps 24 can be interchanged according the particular pull force required to prevent steppage gait or provide the desired flexibility for the wearer. The elastic strap 24 is sufficiently elastic to provide sufficient tensile force to prevent the foot 6 from inadvertently dropping due to foot drop, but has sufficient flexibility to allow the wearer to flex their foot and ankle 6 as needed. As such, a wearer can walk or run using the ankle-foot orthotic 2 with the natural flexing of the foot and ankle 6. The elastic strap 24 further comprises a hook fastener 26 disposed at both the first and second ends 27, 28. The hook fasteners 26 are adapted to engage any looped structure on the wearer's shoes 4 to operably link the ankle brace 8 to the wearer's shoes 4. According to an embodiment of the present invention, the hook fasteners 26 are shaped such that the point of the hook is at least parallel to the shank of the hook or points away from the shank to facilitate easy attachment and removal of the hook from the looped structures on the top of the shoe 4.

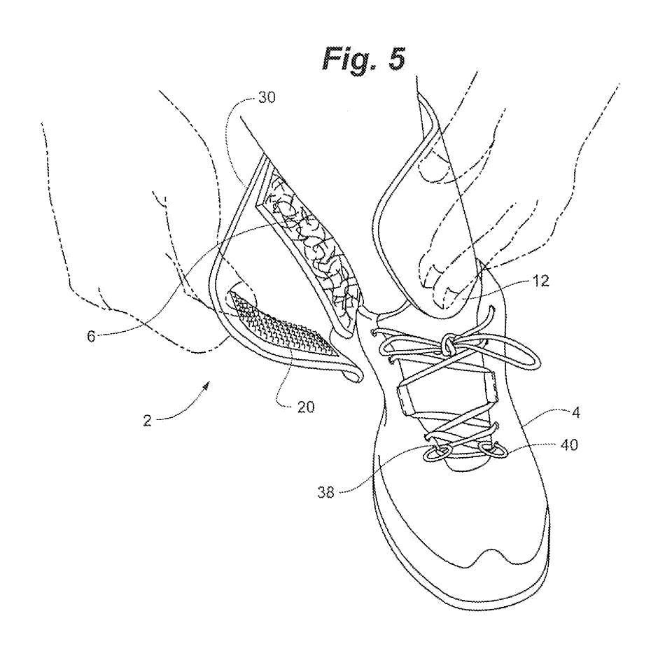

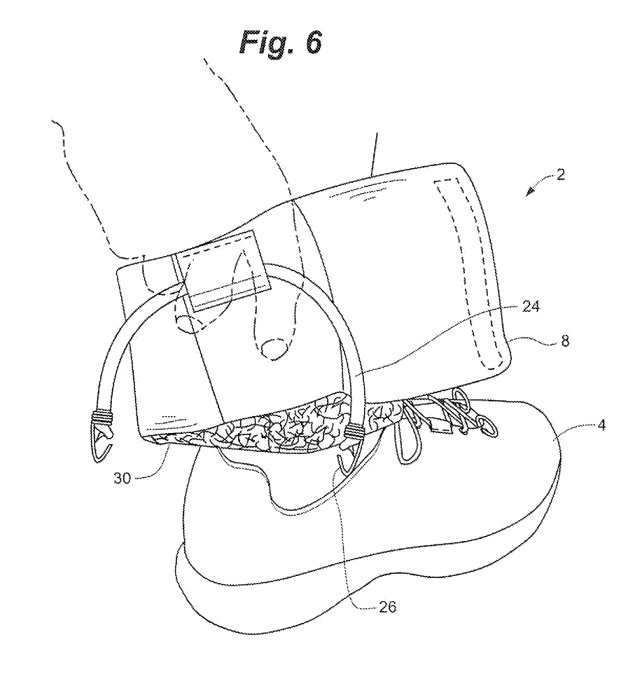

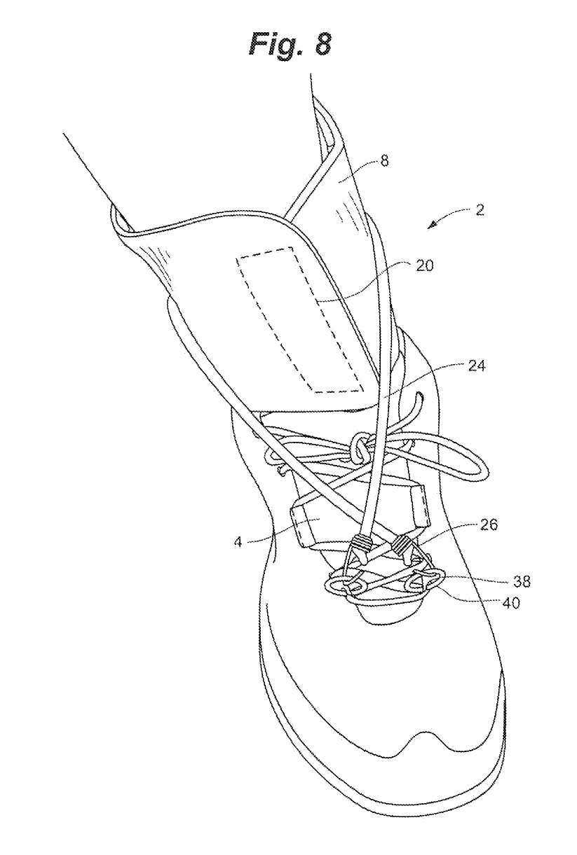

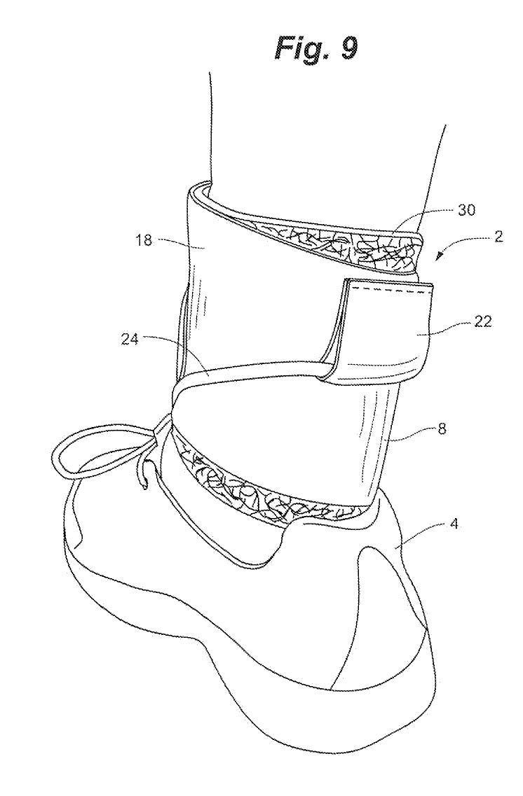

As illustrated in FIGS. 5 to 9, in operation, the first and second wings 12, 14 of the ankle brace 8 are wrapped around the wearer's ankle 6 such that the primary strap loop 22 extends outwardly from the rear of the wearer's ankle 6. In this configuration, the elastic strap 24 is wrapped around the ankle brace 8 and the wearer's ankle 6 before engaging the wearer's shoes 4. Wrapping the elastic strap 24 around the wearer's ankle 6 ensures the correct tension is applied along the elastic strap 24 and properly angles the elastic strap 24 to maintain the wearer's shoe 4 in the proper position. Alternatively, the wings 12, 14 of the ankle brace 8 can be wrapped around the wearer's ankle 6 such that the primary strap loop 22 is positioned at the front of the wearer's ankle 6.

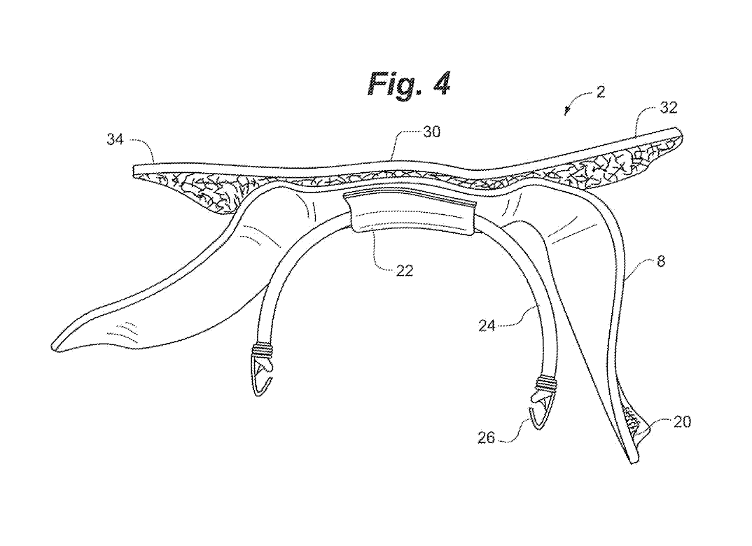

According to an embodiment of the present invention, the ankle brace 8 can further comprise an ankle pad 30 for protecting the wearer's ankle 6 from discomfort. The ankle pad 30 is disposed on the interior face 16 of the ankle brace 8 such that the ankle pad 30 is positioned between the ankle brace 8 and the wearer's ankle 6. The ankle pad 30 can comprise a foam, a mesh or other cushioning material to alleviate wearer discomfort. According to an embodiment of the present invention, the ankle pad 30 can be positioned on the ankle brace 8 such that the ankle pad 30 is positioned against the rear of the wearer's ankle 6 when the ankle brace 8 is wrapped around the wearer's ankle 6. In this configuration, the ankle pad 30 prevents the elastic strap 24 from chaffing or causing other discomfort to the wearer's ankle 6 when the elastic strap 24 is wrapped around the wearer's ankle 6 to engage the wearer's shoe 4. Alternatively, the ankle pad 30 can further comprise a first wing 32 and a second wing 34 (FIG. 4) adapted to wrap around the wearer's ankle 6 to protect both the sides and rear of the wearer's ankle 6 from discomfort.

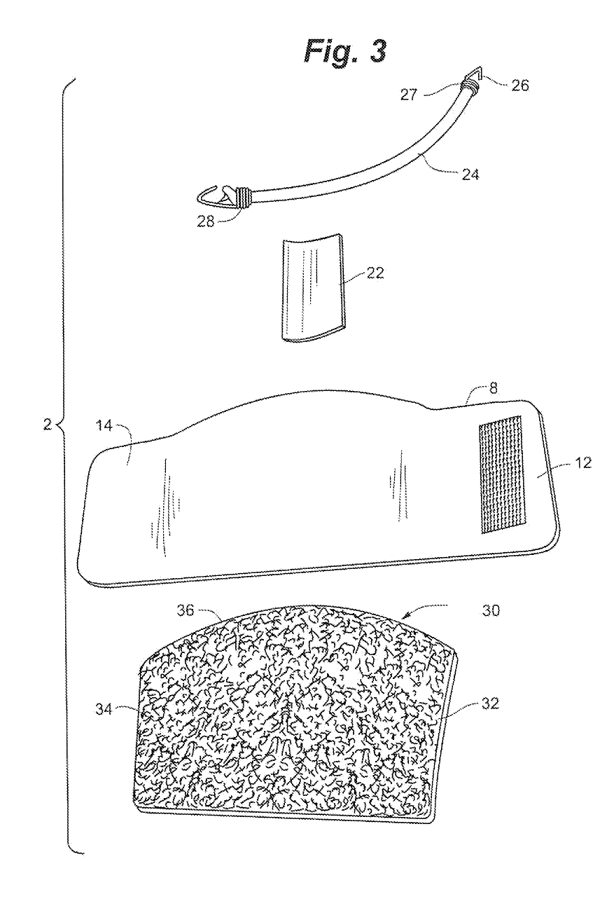

The ankle pad 30 can further comprise a shoe cushion 36 (FIG. 3) for preventing chaffing between the wearer's shoe 4 and the ankle pad 30. The shoe cushion 36 similarly supports the ankle pad 30 to prevent the ankle brace 8 from sliding down the wearer's ankle 6 during use.

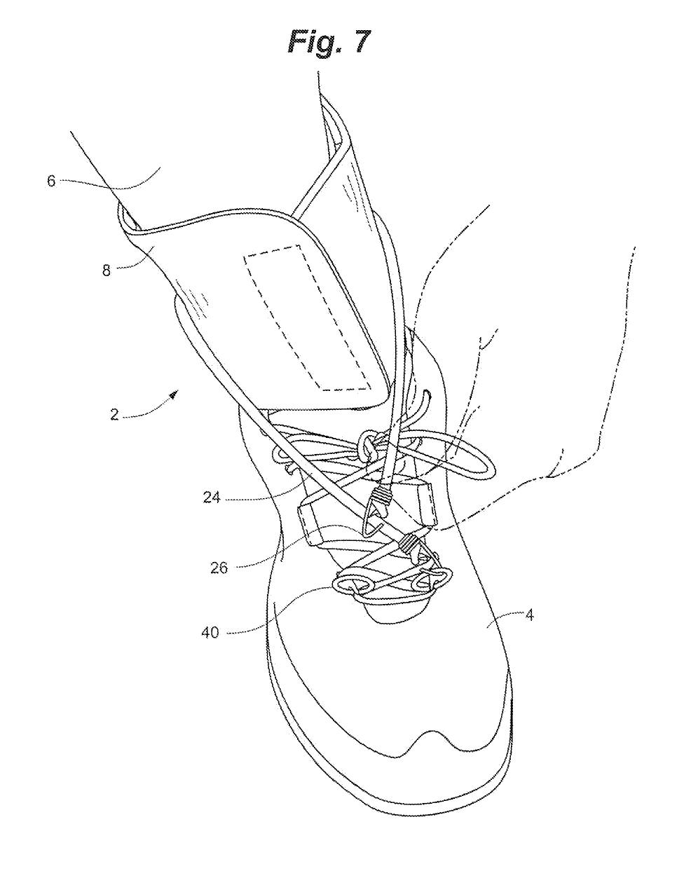

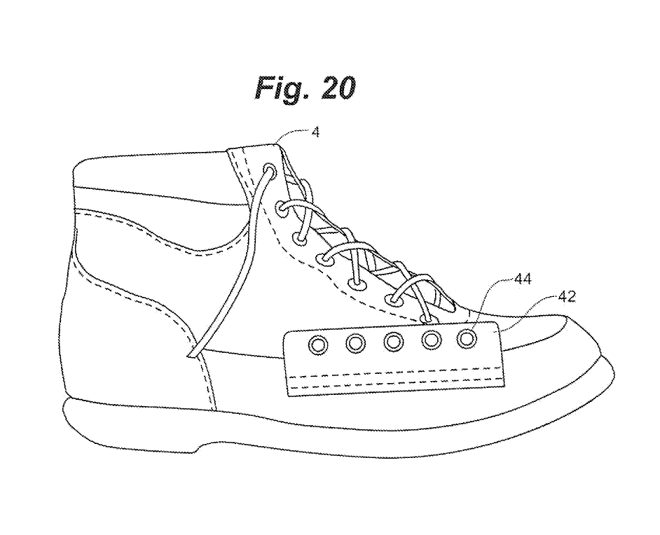

Referring to FIGS. 7 to 9 and 20, the elastic strap 24 comprises hook fasteners 26 for engaging any looped structure on the wearer's shoe 4. Typical shoes 4 can comprise eyelets 38 for receiving shoes laces. The hook fasteners 26 can engage either the laces, eyelets 38 or any other looped structure commonly found on conventional shoes 4. According to an embodiment of the present invention, at least one ring anchor 40 that is easily engageable by the hook fasteners 26 can be threaded through at least one of the eyelets 38. Alternatively, at least one grommet assembly 42 comprising a plurality of grommets 44 releasably engageable by the hook fasteners 26 can be affixed to the shoe 4. The grommet assembly 42 can be sewn, riveted or adhered with adhesive to the user's shoe or other conventional means of affixing the grommet assembly 42. According to an embodiment of the present invention, the grommet assembly 42 can comprise a fabric or leather construction to flex with the user's shoe 4 during walking or running.

As shown in FIGS. 7 to 9 and 20, wearers can customize the arrangement of the elastic strap 24 to accurately direct the tension force applied by the elastic strap 24 and maximize the effectiveness of the ankle foot orthotic 2. Foot drop can affect the various foot muscles differently from wearer to wearer and even from foot to foot, which can cause each afflicted foot to fall uniquely. For example, an afflicted foot can fall downwardly, roll or some combination thereof. As depicted, ring anchors 40 are positioned in only two of the eyelets 38, but can be positioned in each of the eyelets 38. A wearer can mix and match which ring anchor 40 to engage with the hook fasteners 26. For example, a wearer can engage both hook fasteners 26 to the same ring anchor 40 to maximize the force applied to the same part of the shoe 4. Similarly, the hook fasteners 26 can be positioned such that the elastic strap 24 crosses over the top of the shoe 4 or is uncrossed to properly direct the tension force.

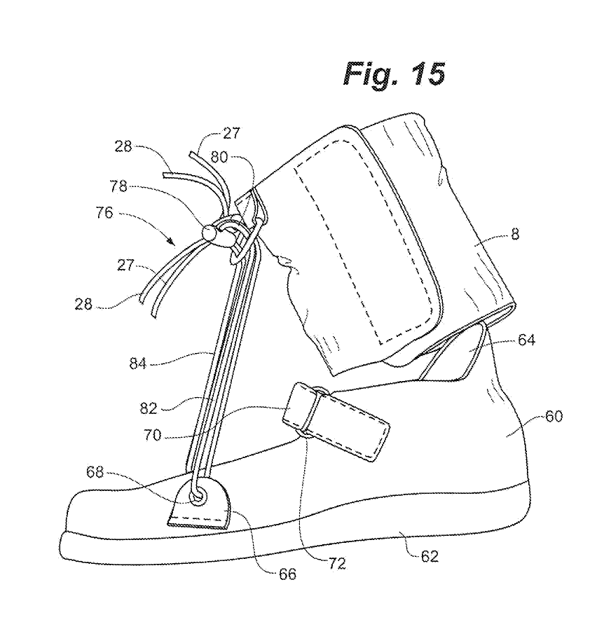

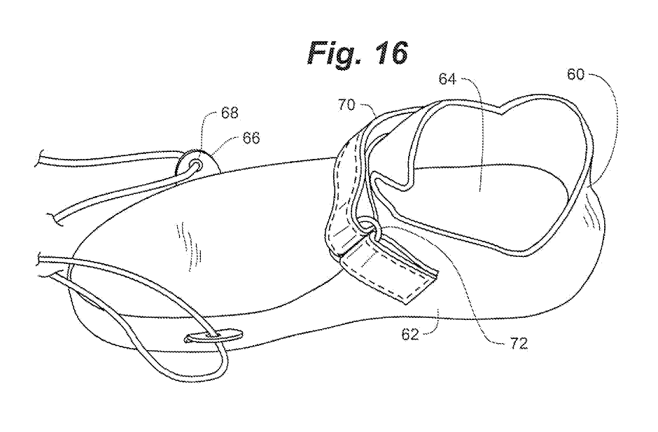

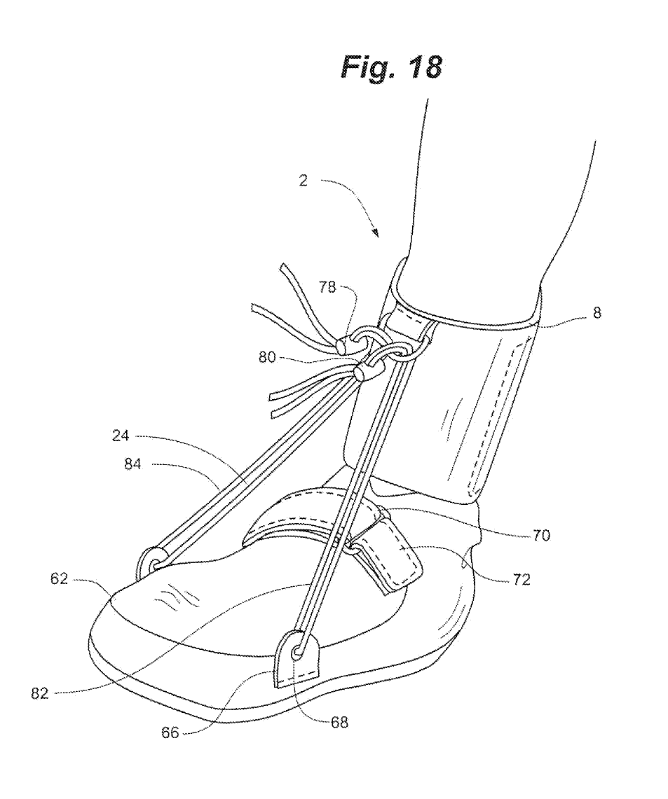

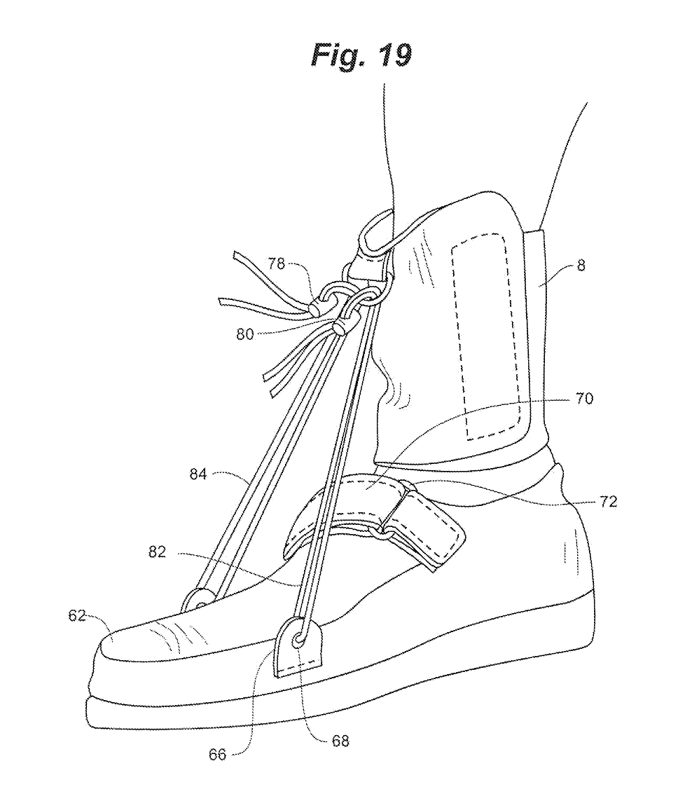

Alternatively, as shown in FIGS. 15 to 19, the ankle brace 8 can be engaged to a slipper 60 according to an embodiment of the present invention instead of a conventional shoe 4. The slipper 60 comprises a slipper body 62 defining an internal volume 64 for receiving the wearer's foot and at least two tabs 66. Each tab 66 comprises an integrated ring 68 releasably engageable by hook fasteners 26. The slipper 60 can further comprise an adjustable strap 70 for adjusting the fit of the slipper body 60. The adjustable strap 70 further comprises a buckle 72 and a strap fastener 74. The adjustable strap 70 is threadable through the buckle 72 and removably adhered to itself with the strap fastener 74. The fit of the slipper 60 can be adjusted by adjusting the length of the adjustable strap 70 threaded through the buckle 72.

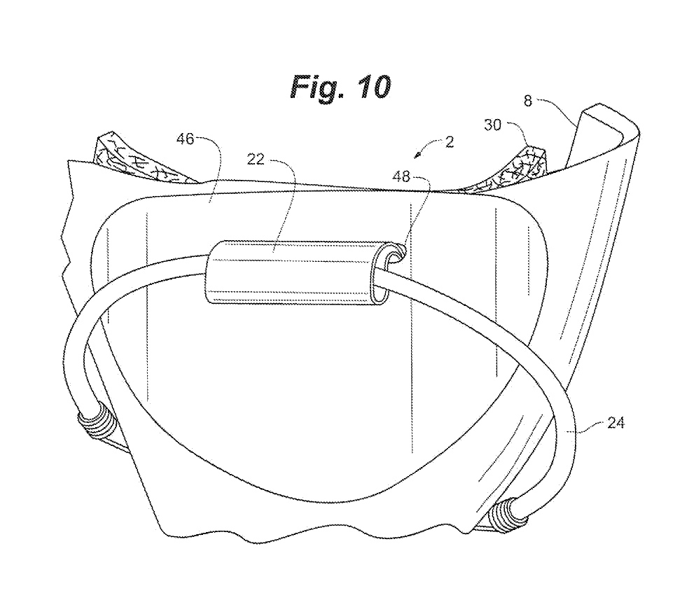

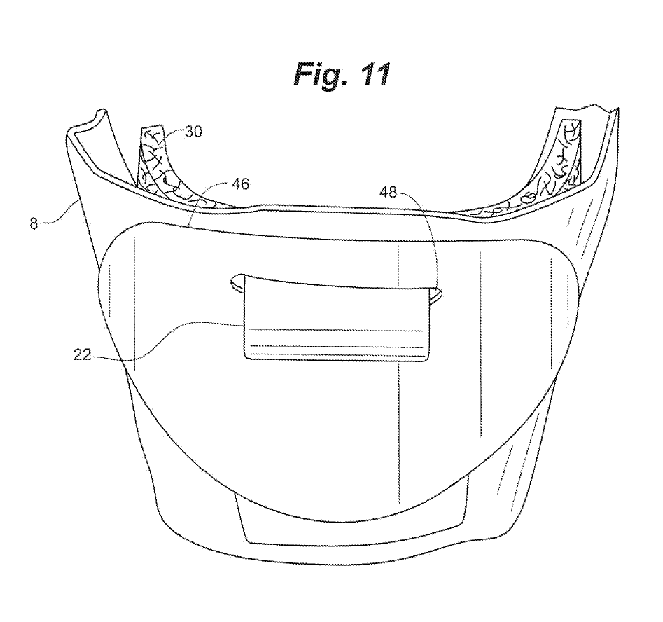

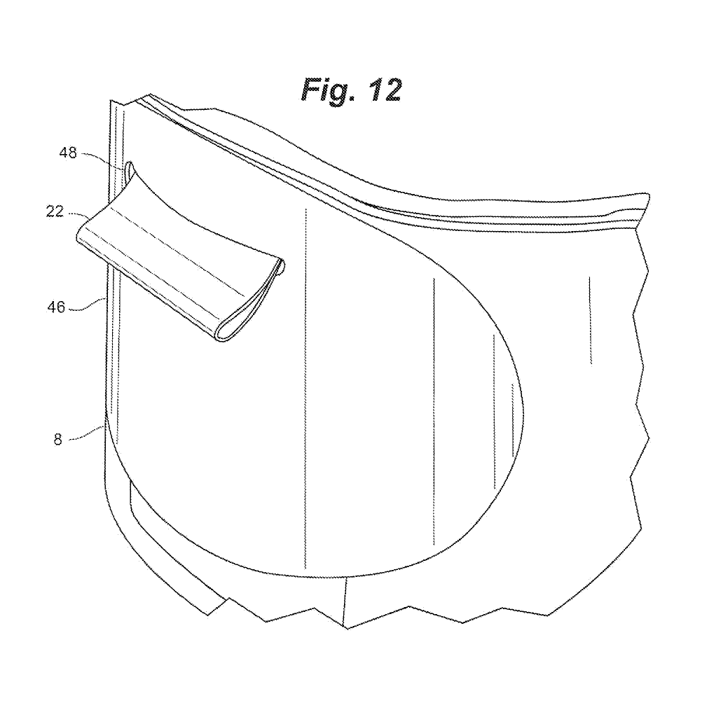

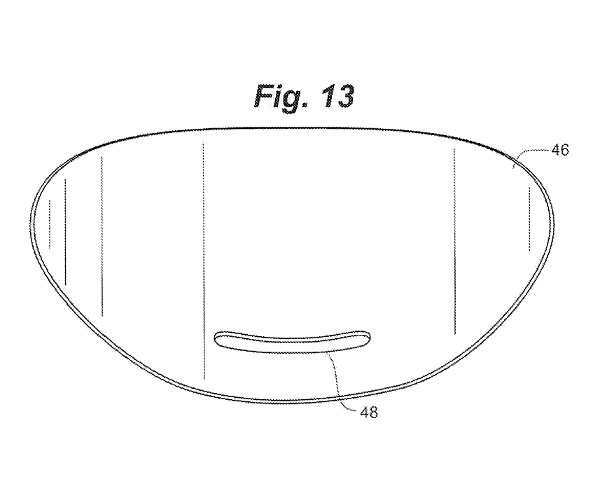

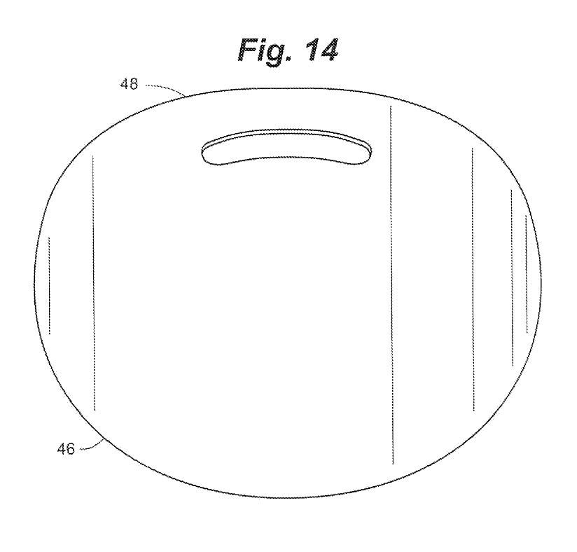

As shown in FIGS. 10-14, an embodiment of the ankle-foot orthotic 2 further comprises a support 46 affixable to the exterior face 18 of the ankle brace 8. The support 46 defines a cutout 48 for receiving the primary strap loop 22 such that the primary strap loop 22 protrudes through the support 46. The elastic strap 24 is insertable through the protruding primary strap loop 22 such that the elastic strap 24 is stretched around the exterior of the support 46 to provide an additional layer between the elastic strap 24 and wearer's ankle 6. The support 46 can comprise a rigid polymer material to provide additional protection and support for the user's ankle 6. According to an embodiment of the present invention, the support 46 is permanently affixed to the ankle brace 8. The support 46 can be retained against the ankle brace 8 by the elastic strap 24 or affixed to the ankle brace 8 by stitching or adhesives.

The support 46 can also comprise a contoured shape to fit the user's ankle 6 to provide additional support for the user during movement and to ensure the ankle-foot orthotic 2 closely follows the user's ankle 6. Similarly, the lower portion of the support 46 can be shaped to prevent the support 46 from hindering the user's movement or flexibility.

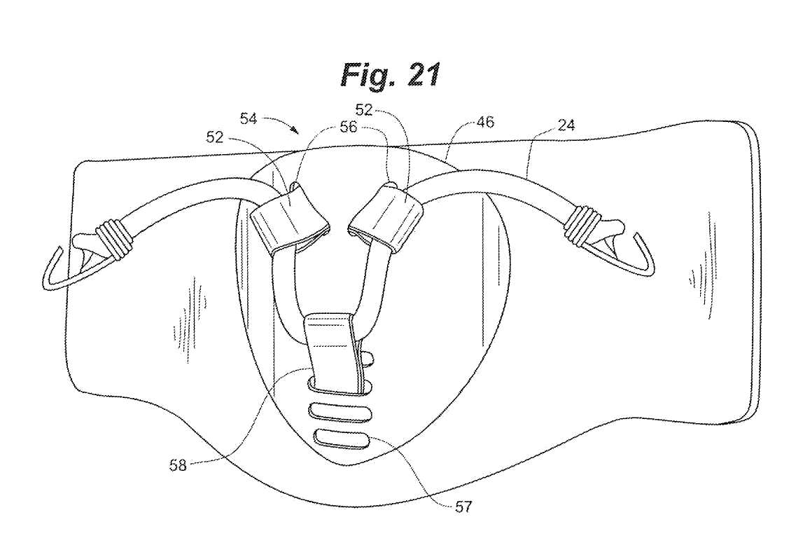

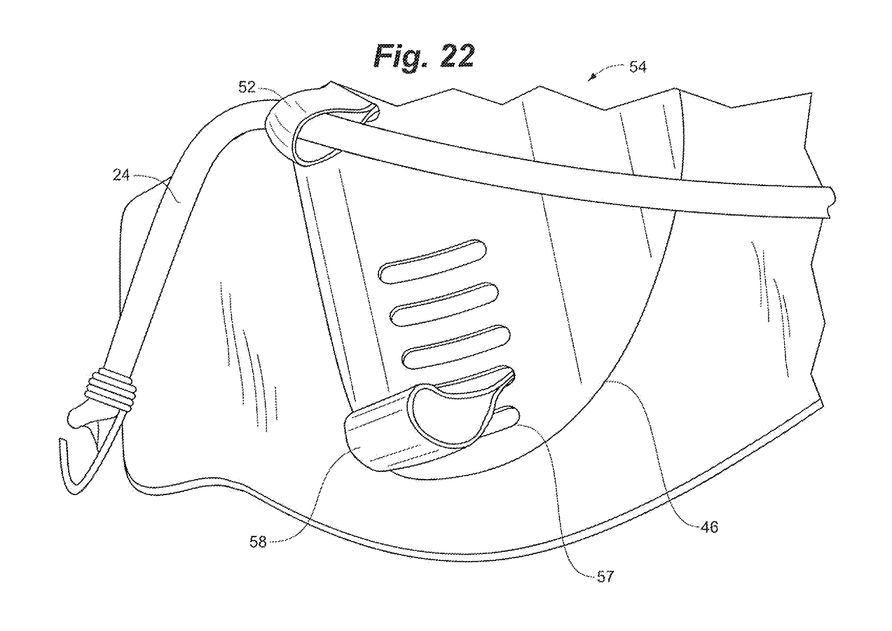

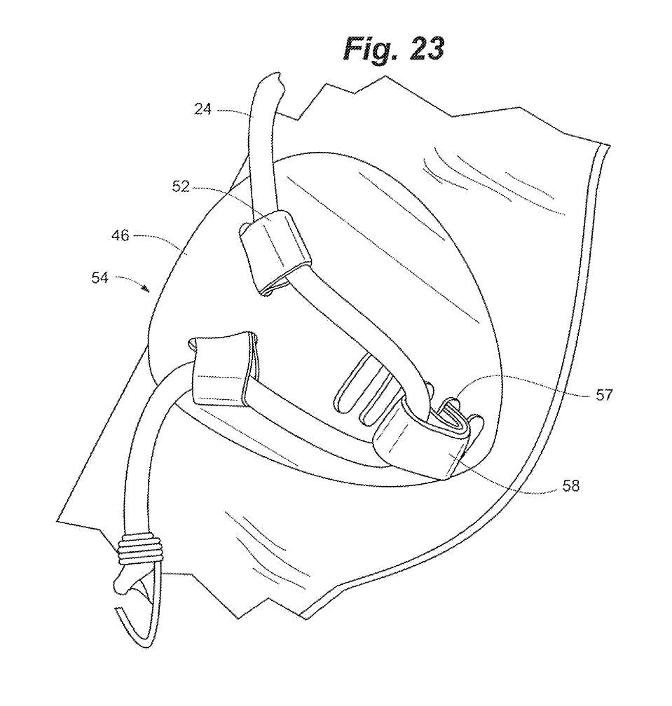

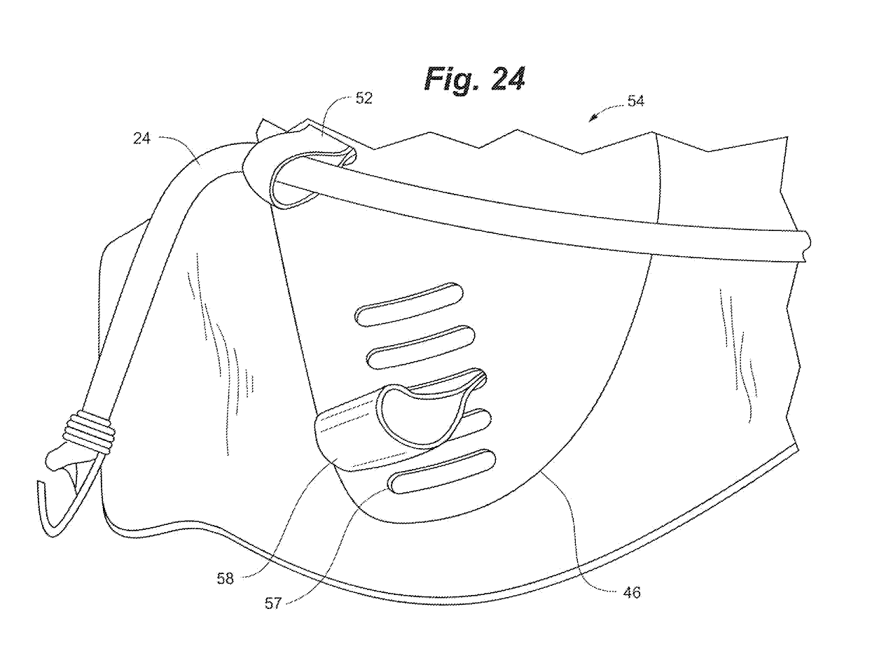

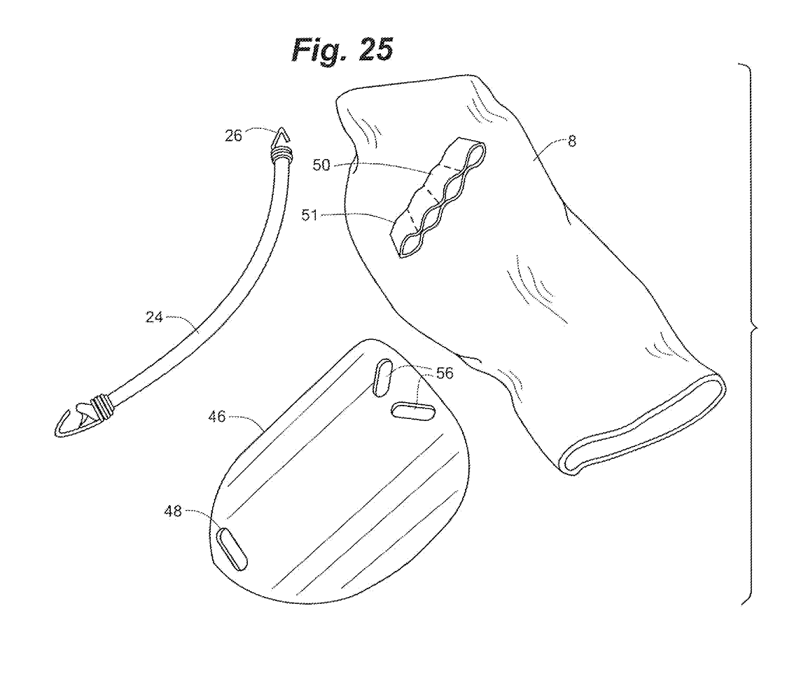

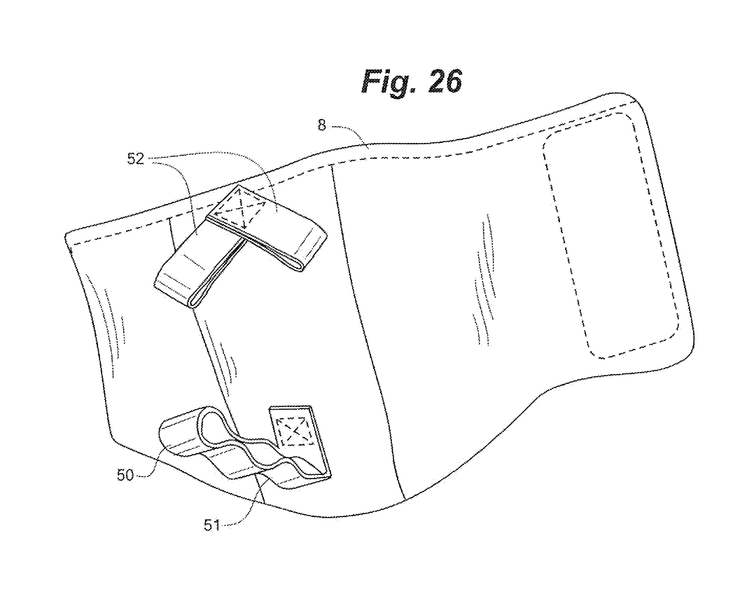

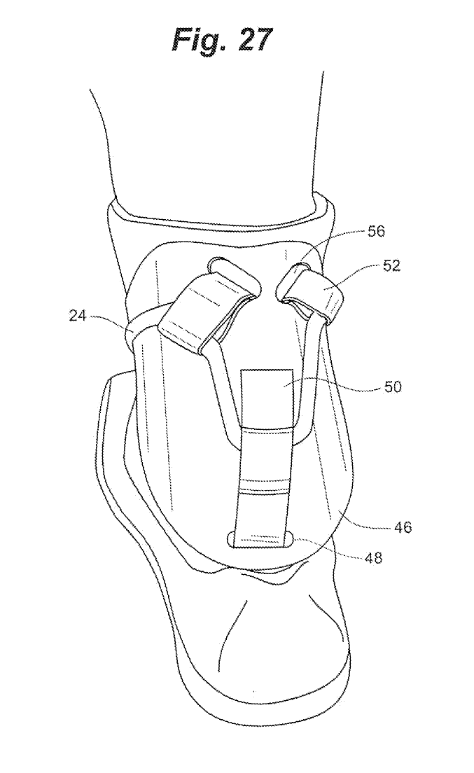

As shown in FIGS. 21 to 27, the support 46 can further comprise a tension adjustment assembly 54 for adjusting the effective length of the elastic strap 24. The effective length of the elastic strap 24 is the portion of the elastic strap 24 extending from the rear of the wearer's ankle 6 to the wearer's shoe 4. The tension adjustment assembly 54 comprises a multi-loop primary strap loop 50 and two secondary strap loops 52 for receiving the elastic strap 24. As shown in FIGS. 25 to 27, primary strap loop 50 comprises a plurality of sub-loops 51. The primary strap loop 50 and the secondary strap loops 52 are affixed the exterior face 18 of the ankle brace 8. In this configuration, the support 46 comprises a primary cutout 48 for receiving the primary strap loop 50 and two secondary strap cutouts 56 for receiving the secondary strap loops 52.

In operation, the primary strap loop 50 and secondary strap loops 52 bend the elastic strap 24 into a u-shape within the tension adjustment assembly 54. Threading the elastic strap 24 through the various subloops 52 of the primary strap loop 50 changes the size of the u-shaped portion of the elastic strap 24 and changing the effective length of the elastic strap 24. Changing the effective length of the elastic strap 24 changes the tension force applied by the elastic strap 24.

As shown in FIGS. 21 to 24, the support 46 can alternatively comprise a plurality of primary cutouts 57 for receiving a fixed length primary strap loop 58. In this configuration, threading the primary strap loop 58 through the various primary cutouts 57 changes the size of the u-shaped portion and correspondingly the effective length of the elastic strap 24.

As shown in FIGS. 15 to 19, the elastic strap 24 can further comprise a strap adjustment assembly 76 comprising a first buckle 78 and a second buckle 80. In this configuration, the first and second ends 27, 28 are threaded through either the ring anchor 40 or the integrated ring 68 and folded back over the elastic strap 24 to form a first loop 82 and second loop 84. The first buckle 78 is positioned on the elastic strap 24 and adapted to receive the first end 27. The second buckle 80 is positioned on the elastic strap 24 and adapted to receive the second end 28. Sliding the buckles 78, 80 along the elastic strap 24 changes the effective length of the first and second loops 82, 84. Increasing the sizes of the loops 82, 84 reduces the effective length of the elastic strap 24 and increases the tension on the elastic strap 24, while reducing the sizes of the loops 82, 84 decreases the effective length of the elastic strap 24 and decreases the tension of the elastic strap 24.

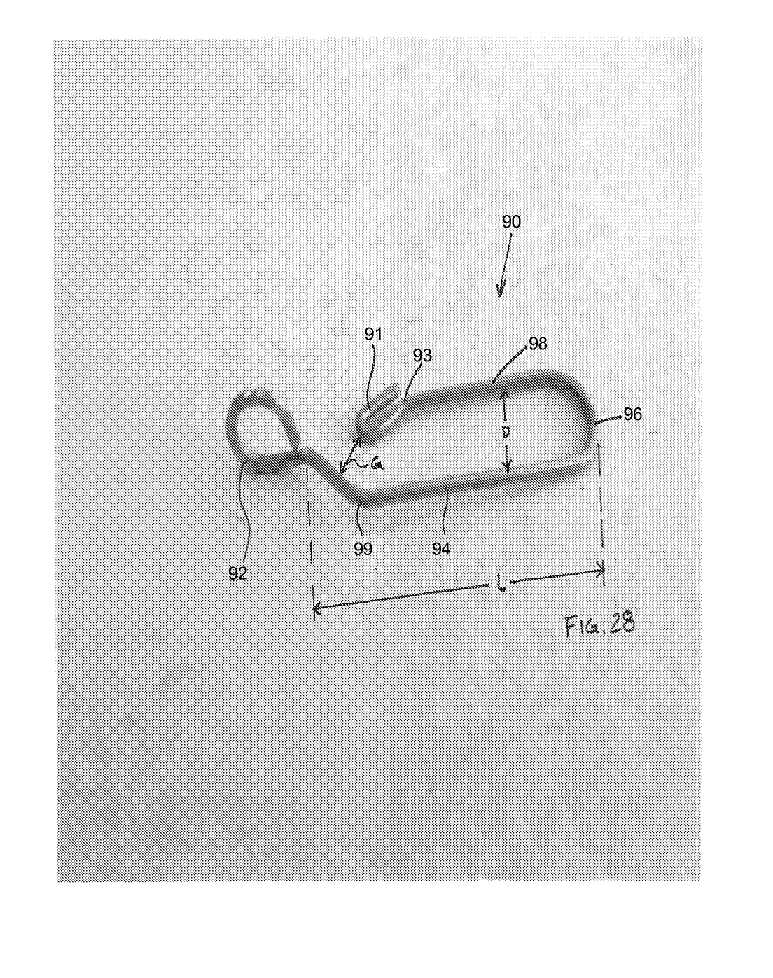

As described above, in reference to FIGS. 7 to 9, in one embodiment, at least one ring anchor 40 is easily engageable by the hook fasteners 26 where the ring anchor 40 can be threaded through at least one of the eyelets 38. It is contemplated that other types of devices can be provided to allow for the hook fastener 26 to be attached to the wearer's shoe so long as the overall function is accomplished. In an exemplary embodiment, a fastener clip can be used. Turning now to FIGS. 28 to 30, at least one fastener clip 90 is easily engageable by the hook fasteners 26 where the fastener clip 90 can be threaded through at least one of the eyelets 38.

As shown in FIG. 28, fastener clip 90 comprises an anchor portion 92, a first longitudinal side portion 94, a curved end portion 96, a second longitudinal side portion 98, and an end portion 91. Fastener clip 90 is configured to be easily inserted and removed from eyelet 38 and has a structure configured to anchor fastener clip 90 in a proper position while allowing rotation and movement of fastener clip 90. Fastener clip 90 can be comprised of a material that is able to be conformed into a desired shape and once conformed is stiff and non-yielding so that the shape is retained, for example, metal.

Anchor portion 92 is configured to provide a dimension or area larger than that of eyelet 38 so that anchor portion 92 cannot be threaded through eyelet 38. First longitudinal side portion 94 and second longitudinal side portion 98 are substantially parallel to each other and are connected via curved end portion 96. In an embodiment, first longitudinal portion 94 can be substantially straight. In another embodiment, first longitudinal portion 94 can be configured to have at least one bend 99. First longitudinal portion 94 has a length L where the length L can be determined based on the configuration of the wearer's shoe.

First longitudinal side portion 94 and second longitudinal side portion 98 are a distance D from each other that is sufficient for the material of the wearer's shoe to be encompassed within. End portion 91 connects to second longitudinal portion 98 at an end 93. In an embodiment, end portion 91 can be configured in a “U” shape. In other embodiments, end portion 80 can be configured in “L” or “V” shapes or be provided with no bends or curves, i.e., straight or substantially straight. First longitudinal side portion 94 and end portion 91 are provided such that a gap G exists between the two portions. The gap G is dimensioned to allow material of the wearer's show to fit therebetween.

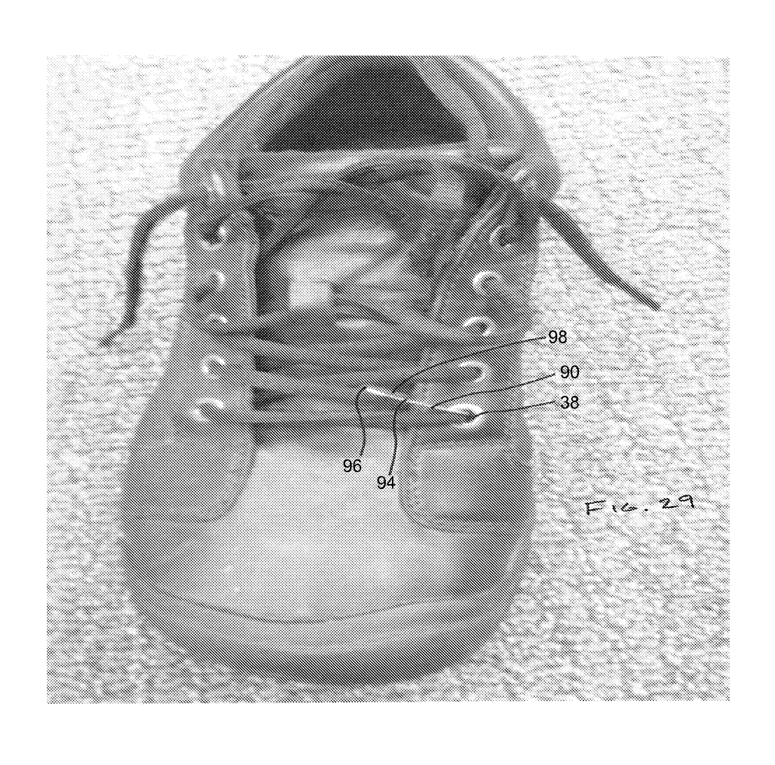

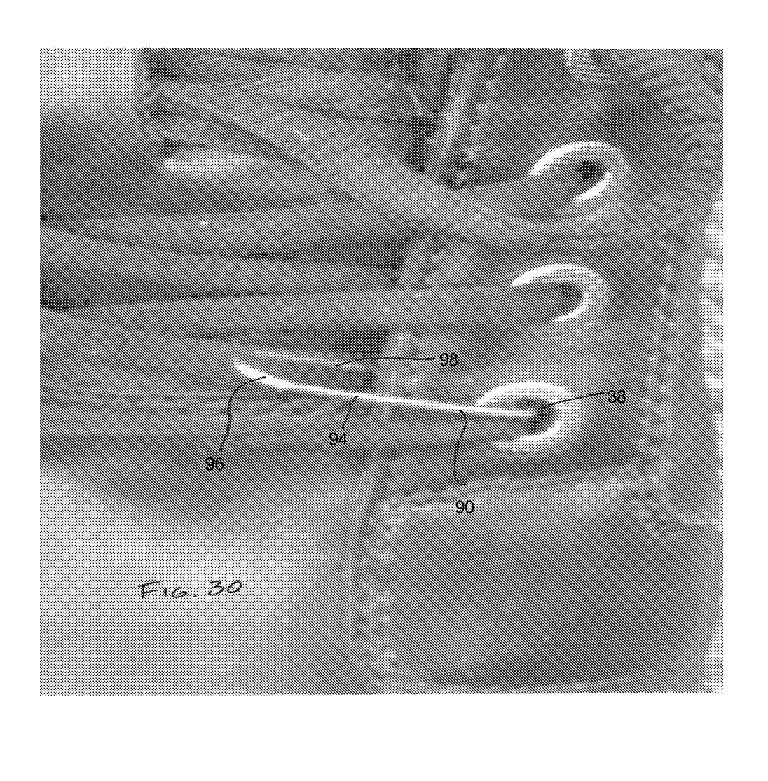

FIGS. 29 and 30 illustrate one embodiment of fastener clip 90 in operational position. In operation, end portion 90 is threaded through eyelet 38. Fastener clip 90 is continually threaded so that second longitudinal side portion 98, then curved end portion 96, and finally first longitudinal side portion 94 move through the eyelet 38 until anchor portion 92 is in contact with eyelet 38. Hook fastener 26 can engage with curved end portion 96 to provide tension force.

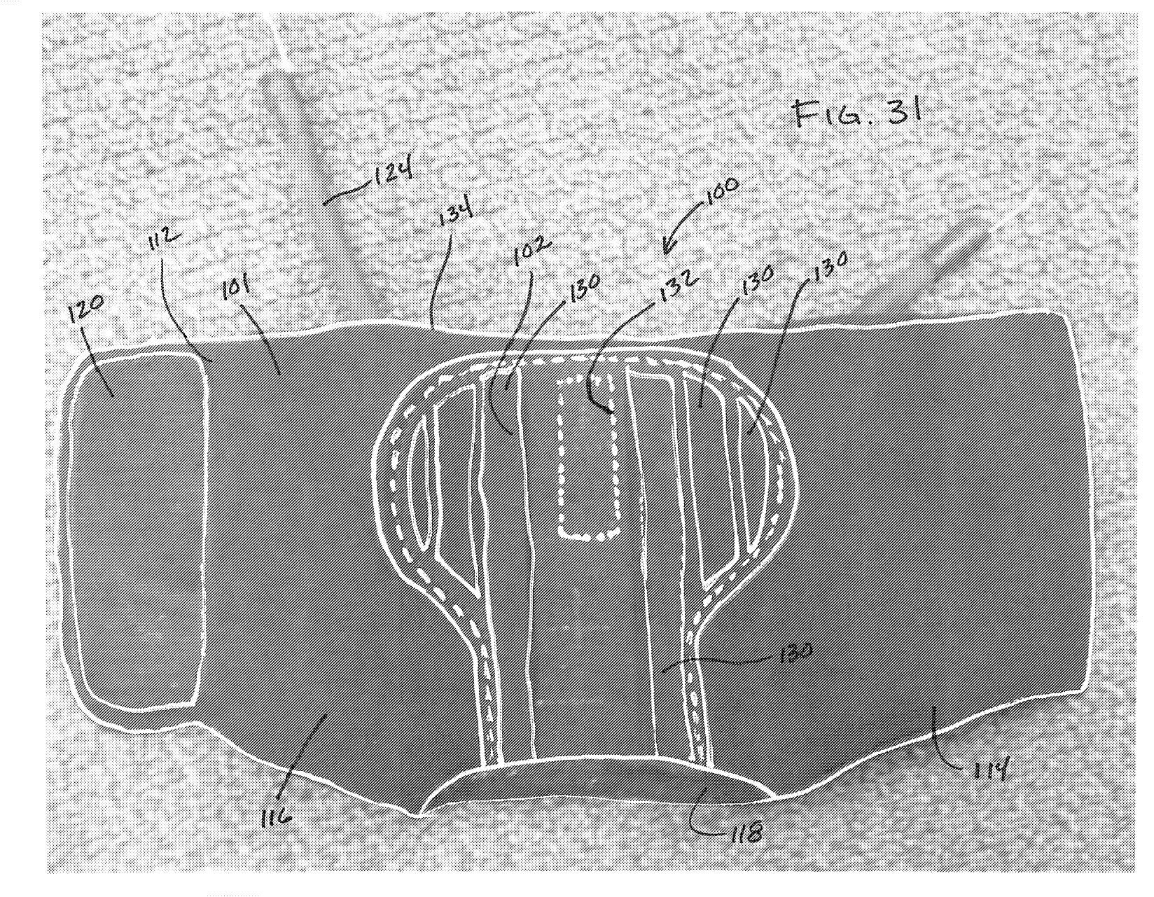

FIG. 31 depicts an embodiment of an ankle-foot orthotic 100 having an ankle brace 101 shaped to conform to the ankle area and having a built-in conformable support 102. The ankle brace 101 comprises a first wing 112 and a second wing 114 and defines an interior face 116 and an exterior face 118 (shown as folded back). Ankle-foot orthotic 100 operates similarly to ankle-foot orthotic 2 (as described in relation to FIGS. 1-5 above) in that ankle brace fastener 120 releasably engages the first wing 112 to the second wing 114 so that the ankle brace 101 forms a generally cylindrical shape around a wearer's ankle and is shaped to conform around the ankle. Similar to ankle brace fastener 20, ankle brace fastener 120 can comprise a VELCRO fastener, an elastic fastener, button fastener or any other temporary fastening means.

Built-in conformable support 102 is fixedly sandwiched between the interior face 116 and the exterior face 118 and is comprised of a plurality of longitudinal support members 130 of varying lengths and configurations. In an embodiment, longitudinal support members 130 can generally define rectangles, quadrilaterals, circular segments, or any combination thereof. Longitudinal support members 130 are comprised of a stiff yet flexible material, for example, a plastic or metal, providing structure to the built-in conformable support 102 while allowing for movement. In an embodiment, longitudinal support members 130 are provided in a group where the longitudinal support members 130 in the group are of different lengths, substantially parallel to each other and separated by a distance from the adjacent longitudinal support member 130. The horizontal width of and distance between the longitudinal support members 130 is sufficient to allow the longitudinal support members 130 to conform around the wearer's ankle area when the ankle-foot orthotic 100 is placed around the ankle.

The vertical length of each individual longitudinal support member 130 is chosen so that when the ankle-foot orthotic 100 is placed around the ankle, the longitudinal support members 130 do not make contact with the lateral malleolus area of the fibula and medial malleolus area of the tibia. A benefit of this is that while the ankle-foot orthotic 100 can be form fitting and provide sufficient structure and support, it still allows mobility of the ankle and reduces pressure points.

In another embodiment, built-in conformable support 102 can be comprised of a unitary support member (not shown) where the unitary support member is manufactured of a stiff yet flexible material that is able to wrap partially around the ankle area while still providing sufficient support. The unitary support member can be molded or shaped so that when in operation, the member does not make contact with the lateral malleolus area of the fibula and medial malleolus area of the tibia.

In an embodiment, a strap loop (not shown) is fixedly attached to the built-in conformable support 102, for example, by stitching the strap loop to the exterior face 118 and the interior face 116 with thread 132 as depicted in FIG. 31. The strap loop can be similar to the strap loop 22 depicted in the ankle-foot orthotic 2 of FIGS. 1 to 5. The strap loop is positioned on the exterior face 118 and is fixedly attached between two of the longitudinal support members 130 so that the strap loop is positioned near the top 134 of the ankle brace 101. An elastic strap 124 is threadable through the strap loop. In another embodiment, the strap loop is fixedly attached to the exterior face 118 only.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. A fastener clip for an ankle-foot orthotic, the ankle-foot orthotic providing resilient support between the leg of a user and a dorsal portion of a user's foot covering, for alleviating foot drop, the ankle-foot orthotic including a brace positionable on the leg of a user, an elastic strap extendable between the brace and dorsal portion of the user's foot covering, the foot covering including structure defining an eyelet extending between opposed surfaces of the foot covering, the eyelet presenting an eyelet width, the fastener clip comprising:
an anchor portion, the anchor portion presenting a minimum anchor portion width that is larger than said eyelet width;
an end portion;
a connecting portion extending between the anchor portion and the end portion, the end portion and the connecting portion each receivable through said eyelet, the connecting portion including spaced apart side portions and a generally curved portion coupling the spaced apart side portions to provide the connection portion with a generally u-shaped appearance,
the end portion and connecting portion of the fastener clip receivable through said eyelet, the anchor portion of the fastener clip engageable with said eyelet for securing said fastener clip in position with said eyelet with said fastener clip curved portion presented for engagement with said elastic strap, thereby providing for the operable, removable engagement of said elastic strap with said user's foot covering.
